(19) United States
(12) Patent Application Publication
(10) Pub. No.: US 2010/0173423 A1
(43) Pub. Date: Jul. 8, 2010

(54) Multiple testing apparatus and method

(75) Inventors: Vivian Zuaretz, Beit Elazari (IL); Falk Fish, Tel-Aviv (IL)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Inverness Medical Switzerland GmbH, Zug (CH)

(21) Appl. No.: 12/319,306

(22) Filed: Jan. 6, 2009

(51) Int. Cl. G01N 33/48

(52) U.S. Cl. 436/164; 422/68.1

(57) ABSTRACT

A multiple test strip assembly comprising a non-bibulous support, a sample application site comprising a defined area of said non-bibulous support delimited by a liquid impervious barrier, and at least two test strips placed on said support, each having a bibulous sample receiving pad, wherein the sample receiving pad of each of said at least two test strips is in contact with said sample application site.

### FIELD OF THE INVENTION

The present invention relates to a multiple testing apparatus and method for concurrently performing two or more strip-format assays on a single sample.

### BACKGROUND OF THE INVENTION

Lateral flow test strips have become increasingly popular over the last thirty years as means for rapid detection of specific constituents in a variety of specimens, including bodily fluids such as urine, saliva, serum, plasma and whole blood. A lateral flow test strip is typically constructed from one or more porous membranes impregnated with assay-specific reagents. To perform a test, a sample is applied to one end of the test strip, referred to as the sample receiving zone. The sample is drawn through the strip by capillary action to pass through a reaction zone where the analyte, when present, reacts with the pre-impregnated assay-specific reagents and then proceeds into a detection zone where the appearance of a visible or otherwise detectable signal indicates presence of the analyte in the sample. Typically, test strips further include an internal procedural control line that is used to validate the test result. Appearance of two lines, therefore, indicates a positive result, while a negative test results in only one line. There exist many variations of test strips, regarding the materials from which the strip is constructed, the distribution and nature of the pre-impregnated reagents and their interaction with the analyte, as well as to the nature and formation of the signal.

Test strips can be used for qualitative or semi-quantitative analysis of many analytes including analytes of clinical interest such as antigens, antibodies, proteins, hormones, enzymes and nucleic acids. The sensitivity and specificity of many available test strips has been shown to be sufficient for clinical diagnostic purposes and therefore they are widely used by medical staff, or as self-testing at home, for rapid diagnosis and therapeutic monitoring of various conditions and disorders.

It is often desired to perform more than one assay on the same sample for detecting two or more independent analytes which indicate two or more different diseases or conditions, or for detecting two or more analytes which are associated with the same disease or condition. For example, it may be desirable to simultaneously detecting an antigen and an antibody which are associated with same pathogen, in order to provide better diagnosis.

However, collected samples can be of a limited volume, thus limiting the number of analyses that can be performed. Moreover, an excess amount of sample is required due to losses such as evaporation and adherence of parts of the sample to the walls of the vessel or of the sample transferring device. Additionally, a major importance in comparing different test results of the same sample is that the tests are performed under the same conditions (e.g. temperature, humidity). Thus, the time of the analysis and the environmental parameters can be of vital importance when comparing the results of various analyses of the same specimen. Consequently, personnel handling parallel tests try to economize and calculate the number of aliquots that can be taken from a sample, and make the best efforts to perform each analysis under the same conditions. However these efforts require high skills and often provide only similar, but not identical, conditions due to the fact that the tests are not done concurrently and with the same aliquot.

There is therefore a need for apparatus and method that will allow for conducting a number of tests on a single sample under identical conditions for improving accuracy of analysis. There is a further need to provide such an apparatus and method that will allow for performing a number of tests on a single sample with minimum loses and in a simple and straightforward manner.

### BRIEF SUMMARY OF THE INVENTION

The present invention provides a multiple testing method and apparatus for analyzing one or more analytes in a sample by using a plurality of test strips. The apparatus comprises at least two test strips overlaid on a non-bibulous planar support and arranged in contact with a common sample application site. The configuration of the multiple testing apparatus provides that subsequently to the application of a sample in the sample application site, portions of the sample are absorbed by the bibulous receiving ends of the strips and are drawn by capillary forces to advance along the strips toward the opposite end thereof.

The common application site is a defined area of the non-bibulous support delimited by a liquid impervious barrier. The liquid impervious barrier may be a line or an area of hydrophobic material affixed to said planar support or, alternatively, may be an elevated wall. The hydrophobic material may be selected from a group consisting of wax, paraffin, oil, crayon, hydrophobic ink, hydrophobic glue or any other hydrophobic material that may be affixed to the planar support. The common application site may be formed as a depression on the non-bibulous support or may be formed by cutting an opening in a non-absorbent film attached onto the planar support.

According to some embodiments, at least a portion of the sample receiving pad of each of the at least two test strips overlaps said sample application site. Yet in accordance with other embodiments, the sample receiving pads of the test strips may be in tangent contact with the sample application site.

The two or more test strips may be arranged in a parallel or a non-parallel configuration. The test strips may be any test strips adapted for detecting an analyte in a liquid sample by a lateral flow assay including qualitative test strips, semi-quantitative test strips and quantitative test strips. The sample may be any liquid sample including bodily liquids such as blood, serum, plasma, urine and bodily secretion.

The invention also relates to a method for concurrently performing two or more test strip assays on a single sample. The method comprises: providing a non-bibulous planar support; placing two or more test strips on the non-bibulous planar support; forming a liquid impervious barrier to define a sample application site in contact with the sample receiving pads of each of the test strips; and applying a sample in said sample receiving site. The method may further comprise adding a reagent solution to the sample application site.

### BRIEF DESCRIPTION OF THE DRAWINGS

The present invention will be understood and appreciated more fully from the following detailed description taken in conjunction with the drawings in which:

FIG. 1 is a top view of a multiple test strip apparatus according to one exemplary embodiment of the present invention;

FIG. 2 is a cross sectional side view of the embodiment shown in FIG. 1 taken along line 2-2;

FIG. 3 is a longitudinal cross sectional side view of another embodiment of the invention;

FIG. 4 is a top view of an exemplary embodiment of the present invention comprising four test strips in contact with a common sample application site;

FIG. 5 is a top view of an exemplary embodiment of the present invention according to which the test strips are arranged on opposite sides of a common sample application site;

FIGS. 6 and 7 are top views of two exemplary embodiments of a dual test strip assembly according to which the sample application site is formed by a hydrophobic barrier;

FIGS. 8 and 9 are top views of a three test strips assembly and a four test strips assembly, respectively.

### DETAILED DESCRIPTION

The present invention discloses a multiple assay apparatus and method for simultaneously performing two or more tests on a single sample by splitting the sample between two or more test strips. The apparatus comprises at least two test strips arranged in contact with a common sample application site. The configuration of the multiple testing apparatus allows for distributing aliquots of the sample between the test strips, thereby sample aliquots advance through the individual strips by capillary forces. Depending on the specific design of the test strips employed, the multiple test results may be visible color signals that can be detected by the naked eye, or alternatively may be read with the aid of an external reading instrument, employing optical reflectance or transmission. The volume of the sample added to the sample application site is measured as the total sum of the individual amounts required for each of the multiple test strips.

The two or more test strips may be identical test strips or different test strips designed for detecting the same analyte, or may be different test strips designed for detecting different analytes. For example, one test strip may be a test strip designed for the detection of an antigen which is associated with a viral or bacterial pathogen and a second test strip may be a test strip designed for the detection of an antibody which is associated with the immunological response to that pathogen.

Referring to FIG. 1, there is shown a first exemplary embodiment of the testing apparatus of the invention, generally designated 10. Apparatus 10 comprises a non-absorbing planar base 50, a non-absorbing film 35 placed on base 50, a sample application site 40 formed as an opening in film 35, and two test strips 20 and 30. Sample application site 40 is formed by cutting out a section of film 35 to expose section 41 of base 50. Planar base 50 is made from non-absorbing inert material such as polypropylene, polyethylene, a paperboard covered with a non-absorbing nylon and the like. Film 35 is a thin non-absorbing material placed on and attached to planar base 50. Preferably film 35 is a pressure-sensitive adhesive tape. Film 35, with or without adhesive layer, may be made from any non-absorbing material such as polypropylene, polyethylene and the like. Strips 20, 30, are fixedly attached to base 50 and to film 35 by any suitable attaching means such as adhesive, a clip and the like.

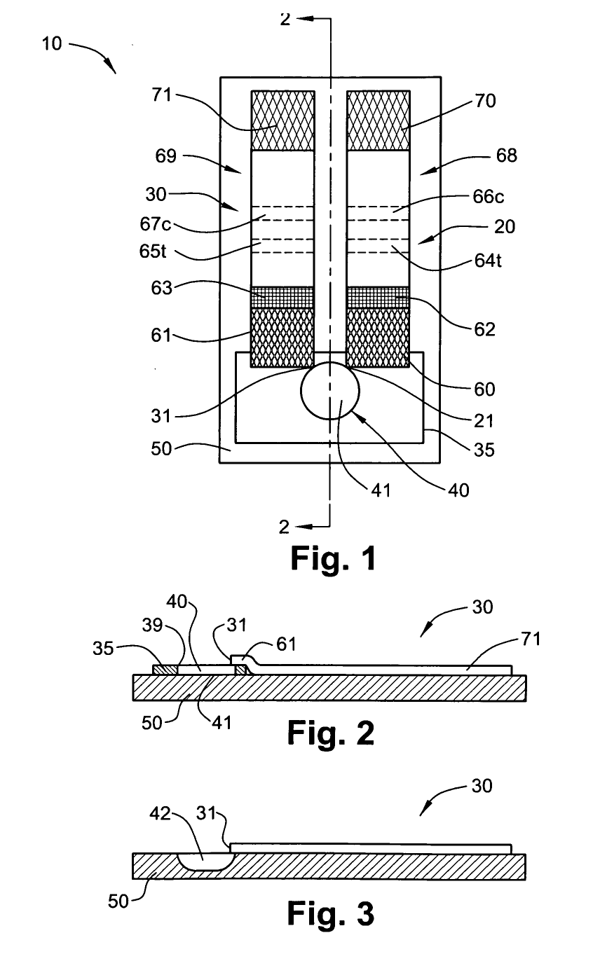

Sample application site 40 is defined by floor 41 of base 50 and by wall 39 which is formed by the cut in film 35, as demonstrated in FIG. 2. Alternatively, the sample application site may be formed as a depression (well) 42, in base 50, as depicted in FIG. 3. In any case, the sample application site is defined by an elevated boundary which prevents liquid placed thereon from spreading beyond the boundary.

Test strips 20 and 30 may be selected to be any test strip known in the art which is designed for detecting an analyte in a liquid sample by a lateral flow assay, including immunoassays, enzymatic assays, biochemical assays and chemical assays. Test strips 20 and 30 comprise a sample receiving pad 60, 61, a reaction zone 62, 63, a detection zone 68, 69 and absorbent wick 70, 71, respectively. Detection zones 68, 69 comprise a test signal line 64t, 65t and a control line 66c, 67c respectively. Test strips 20, 30 may be constructed from one or more bibulous or non-bibulous porous solid phase materials arranged sequentially in an abutting or partial overlapping manner to form a fluid communication therebetween. The strips may be supported on a backing support and/or laminated between two impermeable non-absorbing films such as mylar films, at least one of which is transparent or translucent for allowing viewing the signal. It will be realized that the particular structure of the test strips shown here is given by way of illustration only and that other test strips of different structures may be used without departing from the scope of the present invention.

Strips 20 and 30 are placed on base 50 and partially on film 35 such that one corner 21, 31 of the test strip's sample receiving end overlaps, or is in tangent contact with, sample application site 40. Thus when a liquid sample is added to sample application site 40, the liquid cannot spread beyond wall 39 but can only be absorbed by the test strips. In other words, wall 39 prevents the liquid from advancing to any direction except toward bibulous test strips 20 and 30. The sample is thus spontaneously taken by the naturally hydrophilic individual test strips to advance by capillary forces from the sample receiving end 60, 61 toward wick 70, 71.

To perform a test, a liquid sample is added to sample application site 40, thereafter portions of the sample are drawn concurrently by capillary action into test strips 20 and 30. Thus, the sample is divided between the two test strips and each respective portion advances from the respective sample receiving end 60, 61 through respective reaction zones 62, 63 and further through respective detection zone 68, 69, to opposite respective end 70, 71. The required volume of the sample is substantially the total sum of the individual amounts required for each of the multiple test strips. If an additional reagent solution is required in order to perform the test, the required solution may be added to sample application site 40 before or after the sample as required by the test procedure. Thus, apparatus 10 provides for concurrently running two tests on the same sample under identical conditions. The concurrent tests provide for reduced labor, reduced sample waste and ensures that the tests are actually parallel, thus minimizing the effect of external interfering factors that can influence parallel test analysis.

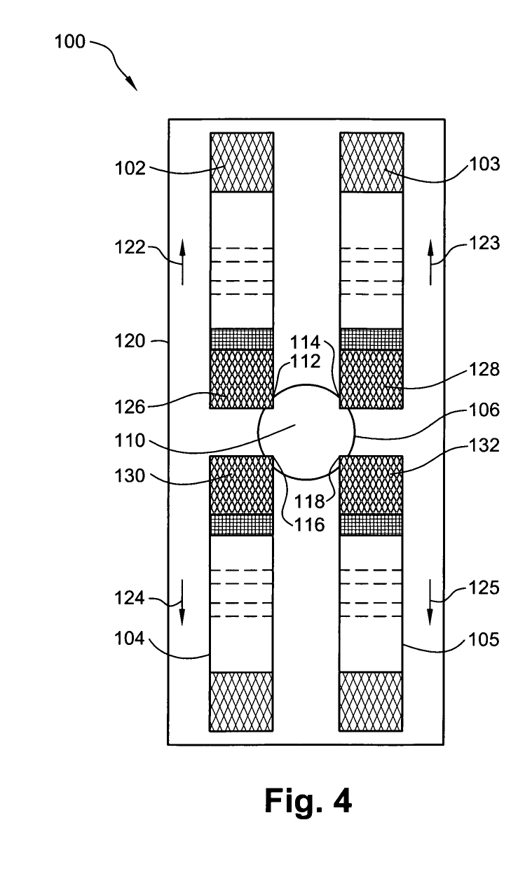

FIG. 4 depicts another embodiment of a multiple test apparatus, designated 100, according to which four test strips 102, 103, 104 and 105 are placed on a planar non-absorbing base 120 in contact with a common application site 110 defined by boundary 106. Sample application site 110 may be formed as an opening in a film attached to base 120 as described above in association with FIGS. 1 and 2, or may be a depression in base 120 as described above in association with FIG. 3. Test strips 102, 103, 104, 105 are placed on base 120 such that one corner 112, 114, 116, 118 of the test strip's sample receiving pad 126, 128, 130, 132, respectively, overlaps sample application site 110. The layout of test strips 102, 103, 104, and 105, provides for a direct flow from the sample application site 110 to the test strips. When a sample is applied on sample application site 110, portions of the sample are absorbed by sample receiving pads 126, 128, 130, 132 and are drawn by capillary forces through the four test strips in the directions indicated by arrows 122, 123, 124 and 125, respectively, to concurrently perform four tests.

It will be realized that the layout of the test strips around the common application site as well as the contact border or contact area between the test strips and the common application site may assume different configurations. Similarly, the shape of the common application site is not limited to a circular shape, but may assume other shapes as well. It will be also realized that the contact area between the individual test strips and the common sample application site is not necessarily the same for all strips. Thus, when different volumes of aliquots are required for different test strips, the contact border or contact area between the individual strips and the sample application site may be manipulated so as to provide the desired ratio between the different aliquots.

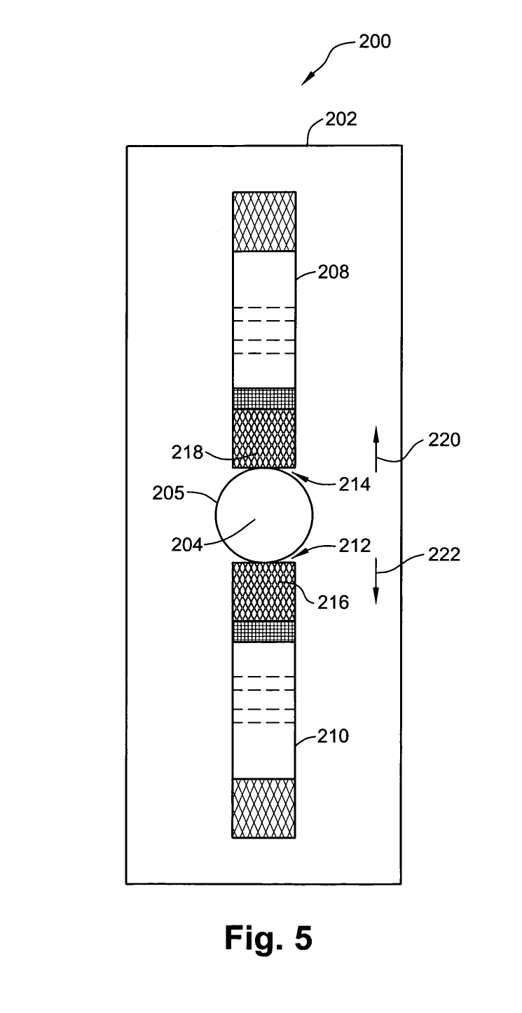

FIG. 5 depicts a dual test apparatus, designated 200, according to which a common application site 204, defined by boundary 205, is located substantially at the center of planar base 202. Two test strips 208 and 210 are placed co-linearly on opposite sides of common application site 204 such that ends 212, 214 of their respective bibulous sample receiving pads 216 and 218 are tangent to boundary 205. Thus, when a sample is added to sample application site 204, portions of the sample concurrently advance in opposite directions through strips 208 and 210, as indicated by arrows 220 and 222.

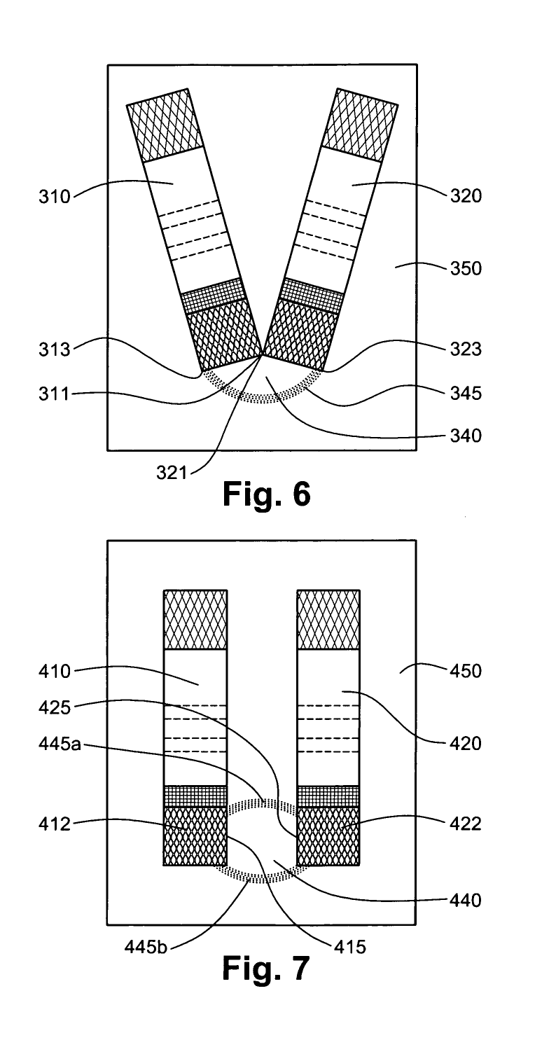

FIG. 6 and 7 illustrate further embodiments of the multiple-testing apparatus of the invention according to which the area of the common application site is defined by a liquid impervious boundary comprising an hydrophobic material which extends between the test strips' sample receiving ends and prevents spreading of liquid in any direction except toward the receiving ends of the test strips. The liquid impervious boundary is formed by applying a line of hydrophobic material on the non-absorbing planar base either before or after the test strips are placed on the base. The hydrophobic material may be any hydrophobic material which can be fixed to the respective non-absorbing base including wax, paraffin, oils, crayon, hydrophobic ink, hydrophobic glue etc., and may be applied to the base by any suitable manner such as printing, stamping, drawing, gluing and the like.

FIG. 6 illustrates a dual testing apparatus comprising a non-absorbing base 350, two test strips 310 and 320 placed on base 350 in a non-parallel layout, and a hydrophobic barrier line 345. In accordance with this embodiment test strips 310 and 320 are attached to base 350 in a ‘V’ shaped layout having their respective corners 311, 321 abutting each other. Barrier line 345 extends between the outer corners 313, 323. A common sample application site 340 is defined by barrier line 345 and respective bibulous ends 313, 323 of test strips 310, 320. When a liquid sample is applied on sample application site 340, the liquid is absorbed by bibulous ends 313, 323 and advance by capillary forces to travel through test strips 310, 320. FIG. 7 illustrates another configuration of a dual testing apparatus according to which the two test strips, 410 and 420, are placed in parallel on non-absorbing base 450. According to this embodiment, two hydrophobic barrier lines 445a and 445b which extend between opposing lateral sides 415, 425 of the test strips' bibulous receiving ends 412, 422, define a common sample application site 440.

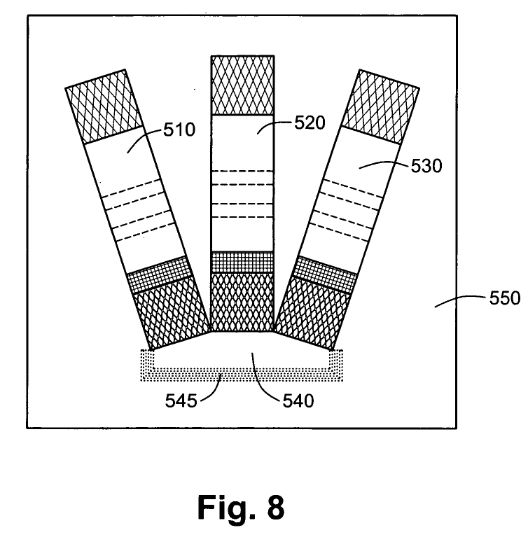

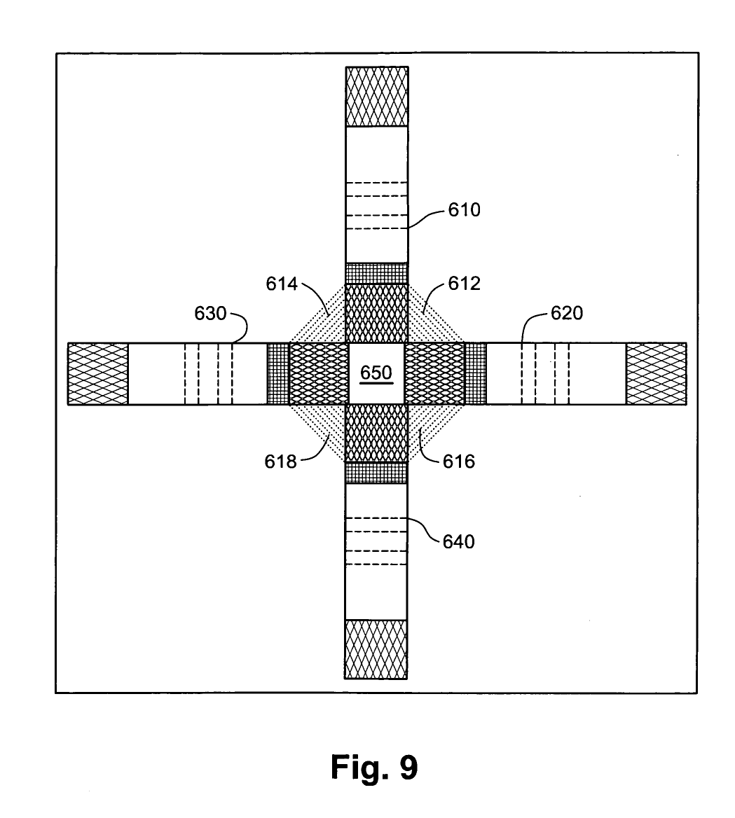

FIGS. 8 and 9 illustrate additional exemplary embodiments according to which more than two test strips share a common application site to enable the simultaneous performance of more than two assays on a single sample. In the embodiment demonstrated in FIG. 8, three test strips 510, 520 and 530 are placed on base 550 in a fan-like layout and a hydrophobic barrier line 545 extends between the outermost corners of the fan to define common sample application site 540. In the embodiment shown in FIG. 9, four test strips 610, 620, 630 and 640 are arranged around a sample application site 650 of a substantially square shape. In accordance with this embodiment, corner areas 612, 614, 616 and 618 are covered by hydrophobic material to prevent spreading of liquid from the sample application site in other directions but the sample receiving pads.

It will be appreciated that the above exemplary embodiments are given for the sake of demonstration only and that many other variations of the present apparatus, regarding the number and layout of the test strips and the outline and area of the common sample application site, may exist without departing from the scope of the present invention.

### EXAMPLE 1

Two Determine® HIV and HBsAg test devices were obtained from Inverness Medical Innovations, Inc., Waltham, Mass., USA. The protective cover was removed from the test devices and the test strips were peeled from the back panel of the devices. The strips were then attached side-by side to the one of the back panels of one of the now empty devices. The distance between the strips was 2 mm. Employing a wax pencil, lines were drawn between the strips at either end of the sample receiving pads of the strips according to the embodiment of FIG. 7 so as to create liquid impervious boundaries between the sample pads. Since each strip is designed for receiving a 50 μL sample, a 100 μL serum sample was placed between the two sample pads. The sample was immediately absorbed by both sample pads and the liquid fronts on both strips advanced at the same rate throughout the 15 minutes test time. Thus, it could be inferred that each of the strips received an equal volume of the sample fluid. The experiment was repeated several times with serum samples and whole blood samples.

### EXAMPLE 2

As in Example 1, two Determine® strips were peeled off from their devices. A 5mm diameter hole was created in a 10×10 mm piece of polyethylene film with adhesive backing. The piece was attached to the back panel of the now empty Determine® device and the two strips were attached on top of the hole according to the embodiment of FIG. 1. As detailed in Example 1, a 100 μL sample was placed in between the two strips and the results were identical, namely the sample was divided equally between the two strips and advanced along the strip at the same rate.

Persons skilled in the art will appreciate that the present invention is not limited to what has been particularly shown and described hereinabove. Rather the scope of the present invention is defined only by the claims, which follow.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A multiple test strip assembly comprising:
a non-bibulous support;
a sample application site comprising a defined area of said non-bibulous support delimited by a liquid impervious barrier; and at least two test strips placed on said support, each having a bibulous sample receiving pad;
wherein the sample receiving pad of each of said at least two test strips is in contact with said sample application site.

2. The multiple test strip assembly of claim 1 wherein at least a portion of the sample receiving pad of each of said at least two test strips overlaps said sample application site.

3. The multiple test strip assembly of claim 1 wherein the sample receiving pad of each of said at least two test strips is in tangent contact with said sample application site

4. The multiple test strip assembly of claim 1 wherein said liquid impervious barrier is a line or an area of hydrophobic material affixed to said planar support.

5. The multiple test strip assembly of claim 4 wherein said hydrophobic material is selected from a group consisting of wax, paraffin, oil, crayon, hydrophobic ink and hydrophobic glue.

6. The multiple test strip assembly of claim 1 wherein the sample application site comprises a liquid impervious floor surrounded by an elevated wall.

7. The multiple test strip assembly of claim 6 wherein the sample application site is a depression in the non-bibulous support.

8. The multiple test strip assembly of claim 6 wherein the sample application site is formed by a cutout in a non-bibulous film attached onto the non-bibulous support.

9. The multiple test strip assembly of claim 1 wherein the two or more test strips are arranged in non-parallel configuration.

10. The multiple test strip assembly of claim 1 wherein the two or more test strips are arranged in a fan-like configuration.

11. The multiple test strip assembly of claim 1 wherein at least one of the at least two test strips is a lateral flow test strip.

12. The multiple test strip assembly of claim 1 wherein at least one of the at least two test strips is a qualitative test strip, a semi-quantitative test strip, or a quantitative test strip.

13. A method for concurrently performing two or more test strip assays on a single sample, the method comprising:
providing a non-bibulous planar support;
placing two or more test strips on said non-bibulous planar support, each of said two or more test strips comprises a sample receiving bibulous pad;
forming a liquid impervious barrier to define a sample application site in contact with the sample receiving pads of each of said two or more test strips, said impervious barrier prevents liquid from spreading in any direction except toward said sample receiving pads; and
applying a sample in said sample receiving site.

14. The method of claim 13 further comprising a step of adding a reagent solution to the sample application site.

15. The method of claim 13 wherein the liquid impervious barrier is formed by fixing hydrophobic material to said planar base.
